(19) United States
(12) Patent Application Publication
(10) Pub. No.: US 2018/0252693 A1
(43) Pub. Date: Sep. 6, 2018

(54) METHODS AND SYSTEMS FOR PREVENTING DAMAGE TO INTERNAL COMPONENTS OF NATURAL GAS VEHICLE GAS DETECTORS

(71) Applicant: Honeywell International Inc., Morris Plains, NJ (US)

(72) Inventors: Ouyang Yang, Shanghai (CN); Tao Chen, Shanghai (CN); Peipei Liu, Nanjing (CN)

(21) Appl. No.: 15/450,426

(22) Filed: Mar. 6, 2017

(51) Int. Cl. G01N 33/22; G01D 11/24; B60H 1/00

(52) U.S. Cl. CPC G01N 33/225; B60H 1/008; G01D 11/245

(57) ABSTRACT

Embodiments relate generally to methods and systems for balancing the internal pressure of a gas detector. A method for venting internal pressure changes of a gas detector may comprise providing an inlet to a sensor located within a housing of the gas detector; isolating the sensor from the rest of the interior of the housing; providing at least one vent between the interior of the housing and the external environment, wherein the sensor is isolated from airflow into and out of the vent; and preventing one or more elements from entering the interior of the housing through the at least one vent via a breathable membrane aligned with the at least one vent.

### CROSS-REFERENCE TO RELATED APPLICATIONS

Not applicable.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

Gas detectors may be employed in various environments to monitor the level of certain gases in the ambient environment. Gas detectors may comprise alarm or alert capabilities, and may be configured to communicate with other devices or systems to notify users of the detected gas levels.

### SUMMARY

In an embodiment, a method for venting internal pressure changes of a gas detector may comprise providing an inlet to a sensor located within a housing of the gas detector; isolating the sensor from the rest of the interior of the housing; providing at least one vent between the interior of the housing and the external environment, wherein the sensor is isolated from airflow into and out of the vent; and preventing one or more elements from entering the interior of the housing through the at least one vent via a breathable membrane aligned with the at least one vent.

In an embodiment, a gas detector may comprise a housing configured to enclose one or more components of the gas detector, the housing comprising an inlet and at least one vent; at least one sensor located within the housing, and in fluid communication with the inlet of the housing, configured to detect one or more characteristics of the ambient environment; a fluid channel configured to isolate the sensor from the at least one vent; and at least one breathable membrane located adjacent to the at least one vent, configured to prevent one or more elements from entering the interior of the housing through the at least one vent, wherein when the internal pressure of the housing increases, airflow is directed through the at least one vent to balance the internal pressure with the external pressure of the housing, and wherein when the internal pressure of the housing decreases, airflow is directed through the at least one vent to balance the internal pressure with the external pressure of the housing.

In an embodiment, a method for venting internal pressure changes of a gas detector may comprise exposing the gas detector to a sensed environment, wherein the gas detector comprises a housing comprising an inlet and at least one vent; at least one sensor in fluid communication with the inlet of the housing; a fluid channel configured to isolate the sensor from the at least one vent; and at least one breathable membrane located adjacent to the at least one vent; preventing one or more elements from entering the interior of the housing through the at least one vent via the breathable membrane; determining one or more characteristics of the sensed environment via the at least one sensor; and when the internal pressure of the housing changes, balancing the change in pressure with the pressure of the external environment via the at least one vent.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 illustrates a schematic drawing of a gas detector according to an embodiment of the disclosure.

FIG. 2 illustrates a cross-sectional view of a gas detector according to an embodiment of the disclosure.

FIG. 3 illustrates an assembled view of a gas detector according to an embodiment of the disclosure.

FIG. 4 illustrates a detailed view of a breathable membrane according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example;

The terms “about” or “approximately” or the like, when used with a number, may mean that specific number, or alternatively, a range in proximity to the specific number, as understood by persons of skill in the art field; and

If the specification states a component or feature “may,” “can,” “could,” “should,” “would,” “preferably,” “possibly,” “typically,” “optionally,” “for example,” “often,” or “might” (or other such language) be included or have a characteristic, that particular component or feature is not required to be included or to have the characteristic. Such component or feature may be optionally included in some embodiments, or it may be excluded.

Embodiments relate to systems and methods for preventing damage to internal components of a gas detector due to high internal pressures. The gas detector may be configured to detect one or more gasses and/or characteristics of the ambient air. The gas detector may be configured to be used in natural gas powered vehicles. Some national and international standards may require natural gas vehicles to contain a gas detector to monitor the levels of natural gas in the ambient environment and therefore vehicle safety. The gas detector may issue safety warnings to a user and/or monitor, for example when a natural gas leak is detected.

Typical gas detectors, particularly natural gas detectors, may not be required to withstand significant changes in temperature and pressure during the use life of the gas detector. However, gas detectors that are used to monitor natural gas power vehicles may be subject to vibrations as well as changes in temperature, pressure, and humidity. These changes in the environment may impact the performance of the gas detector, and could cause damage to the gas detector.

Embodiments of the disclosure include systems and methods for preventing damage to a gas detector due to changes in the environment. The gas detector may comprise one or more vents or ports configured to allow airflow into and out of the housing of the gas detector, thereby balancing the internal pressure of the housing with the external pressure. While described as airflow, only a minor amount of gas exchange may be needed to balance a pressure in the housing, and a bulk airflow is not necessarily needed. The gas detector may comprise one or more breathable membranes located adjacent to any openings in the housing that can be configured to prevent one or more elements from entering the housing of the gas detector and damaging internal components of the gas detector. The breathable membranes may comprise waterproof breathable membranes. The breathable membrane may be located on an inlet to a sensor within the housing of the gas detector, wherein the breathable membrane may prevent elements from reaching the sensor. The breathable membrane may prevent harmful elements from entering the housing while also allowing airflow into and out of the housing. Additionally, gas molecules may pass through the breathable membrane to the sensor.

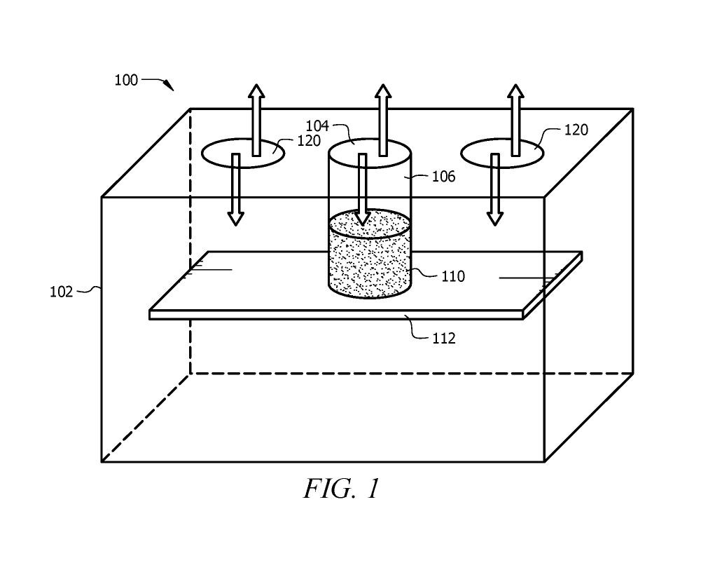

Referring to FIG. 1, a schematic drawing of a gas detector 100 is shown. The gas detector 100 may be a methane gas detector, though other flammable gases or combustible gas mixtures can also be detected. The gas detector 100 may comprise a housing 102 and a sensor 110, wherein ambient air may enter the housing 102 via an opening 104 and interact with the sensor 110. The sensor 110 may located within the housing 102, and may be isolated from the rest of the interior of the housing 102. A fluid channel 106 may connect the sensor 110 to the opening 104. The sensor 110 may also be connected to a controller 112, such as a printed circuit board (PCB). The sensor 110 may be configured to determine a concentration of a particular gas or gases in the ambient air. The sensed concentration may be determined from an output signal from the sensor 110 that is processed by the controller 112.

The housing 102 may comprise a sealed enclosure configured to protect the internal components of the gas detector 100 from the ambient environment. In some embodiments, the housing 102 may comprise a low quantity of aluminum alloy magnesium, which may prevent collision sparks which may be caused by vibration of the housing 102. The pressure within the housing 102 may be affected by the temperature and/or pressure of the ambient environment. Additionally, vibrations and other external forces may affect the pressure within the housing 102. To prevent damage to the internal components, particularly the electrical components, due to changes in pressure within the housing 102, one or more vents 120 may be incorporated into the housing 102. These vents 120 may allow for airflow into and out of the housing 102, so that the pressure within the housing 102 may be balanced.

By balancing the pressure within the housing, the gas detector may be used in high temperature applications, where damage to the gas detector due to pressure variations may be prevented or reduced by the vents 120. The sensor 110 may be isolated from the air that enters and/or exits the housing 102 via the vents 120. In some embodiments, the vents 120 may be located adjacent to the inlet 104. In some embodiments of the gas detector, the vents 120 and the inlet 104 may be located on the same side of the housing 102, so that the housing 102 may be attached to another device or surface without blocking the inlet 104 and/or vents 120.

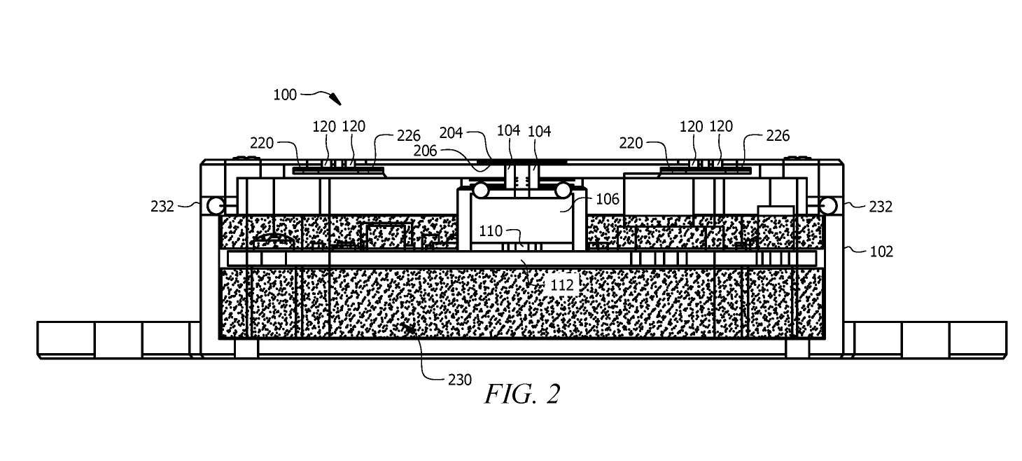

FIG. 2 illustrates a cross-sectional view of the gas detector 100. The gas detector 100 may comprise a breathable membrane 204 located about the inlet 104, wherein the breathable membrane 204 may allow airflow into and/or out of the inlet 104 while preventing particulate matter and/or water from entering the inlet 104 and/or housing 102.

The breathable membrane 204 may be configured to prevent failure of internal components of the gas detector 100 by preventing harmful substances from entering the housing via the inlet 104. Additionally, the breathable membrane 204 may prevent internal condensation within the housing 102. The breathable membrane 204 may be waterproof or water resistant. The breathable membrane 204 may be attached to the housing 102 around the inlet 104 via a layer of adhesive 206. In some embodiments, the adhesive 206 may attach to the edges of the breathable membrane 204, leaving a portion of the breathable membrane 204 free of the adhesive 206. In some embodiments, the airflow may pass through the portion of the breathable membrane 204 that is not contacted by the adhesive 206.

Additionally, the one or more vents 120 may comprise one or more breathable membranes 220 located between the vents 120 and the interior of the housing 102. In some embodiments, the vents 120 may comprise a plurality of openings, wherein each of the openings may align with at least a portion of the breathable membrane(s) 220. In some embodiments, the breathable membrane(s) 220 may be attached to the housing 102 via a layer of adhesive 226. In some embodiments, the adhesive 226 may attach to the edges of the breathable membrane 220, leaving a portion of the breathable membrane 220 free of the adhesive 226. In some embodiments, the airflow may pass through the portion of the breathable membrane 220 that is not contacted by the adhesive 226.

The breathable membranes 204 and 220 may comprise expanded polytetrafluoroethylene (ePTFE) material, which may be water-proof, dust-proof, and water- and oil-repellent. The ePTFE material may comprise a multi-layer microporous ventilated membrane configured to filter harmful substances from entering the housing. By preventing these harmful substances from entering the housing, the breathable membranes 204 and 220 may ensure normal function of the sensor and controller, and improve the accuracy of the sensed data. In some embodiments, the breathable membranes 204 and/or 220 may allow for the gas detector 100 to be classified as IP67 grade water resistant.

The controller 112 of the gas detector 100 may be held in place within the housing 102 via a protection material 230, such as a potting material (e.g., an epoxy material, etc.). The protection material 230 may fill at least a portion of the interior space of the housing 102 around the controller 112, thereby protecting the elements of the controller 112. The protection material 230 may also prevent damage to the internal components of the gas detector 100 due to vibrations. Additionally, the protection material 230 may comprise heat conduction properties allowing heat conduction away from the sensor 110.

In some embodiments, the gas detector may comprise one or more O-rings 232 configured to seal the housing 102, and may act as a secondary water-proofing for the housing 102. Additionally, the O-rings may comprise heat conduction properties allowing heat conduction away from the sensor 110.

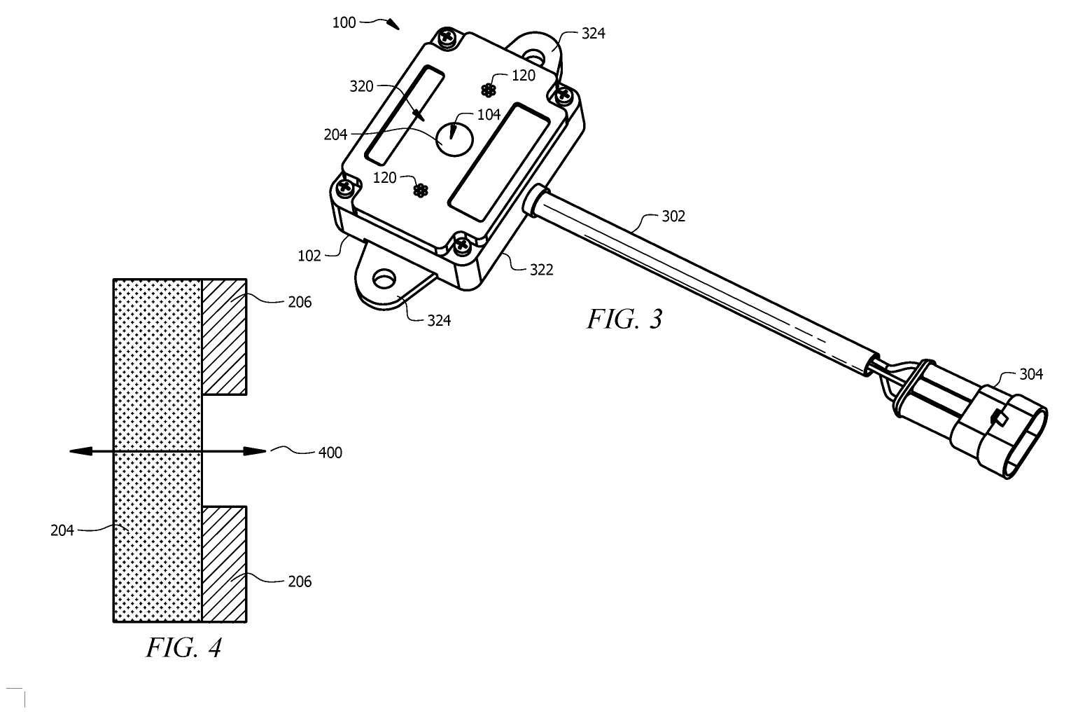

FIG. 3 illustrates an assembled view of the gas detector 100, wherein the housing 102 may be configured to connect to one or more wires 302 and/or a connector 304, where the wires 302 may be in communication with the controller 112 of the gas detector 100, and may be configured to communicate information to and from the gas detector 100. In some embodiments, when the gas detector 100 is employed on or near a natural gas vehicle, the connector 304 may directly connect the gas detector 100 to a communication system within the vehicle.

In some embodiments, the inlet 104 and the one or more vents 120 may be located on a first surface 320 of the housing 102. This may allow for the housing 102 to be attached to another device or surface without blocking airflow to the inlet 104 and the vents 120. For example, a second surface 322 of the housing 102 may be attached to another device or surface via attachment points 324.

FIG. 4 illustrates a cross-sectional view of the breathable membrane 204. The description of the breathable membrane 204 may also apply to the breathable membrane(s) 220. As shown in FIG. 4, the adhesive material 206 may be applied only at the edges of the breathable membrane 204. The placement of the adhesive material 206 may allow for airflow 400 to pass through the breathable membrane 204 without hindrance from the adhesive material 206.

In a first embodiment, a method for venting internal pressure changes of a gas detector may comprise providing an inlet to a sensor located within a housing of the gas detector; isolating the sensor from the rest of the interior of the housing; providing at least one vent between the interior of the housing and the external environment, wherein the sensor is isolated from airflow into and out of the vent; and preventing one or more elements from entering the interior of the housing through the at least one vent via a breathable membrane aligned with the at least one vent.

A second embodiment can include the method of the first embodiment, further comprising employing the gas detector in an increased temperature environment; increasing the internal pressure within the housing due to the increased temperature; and balancing the increased pressure within the housing with the pressure of the external environment via the at least one vent.

A third embodiment can include the method of the first or second embodiments, further comprising employing the gas detector in an increased pressure environment; and balancing the increased pressure within the housing with the pressure of the external environment via the at least one vent.

A fourth embodiment can include the method of any of the first to third embodiments, further comprising locating the at least one vent and the inlet on a first surface of the housing.

A fifth embodiment can include the method of the fourth embodiment, wherein a second surface of the housing is attached to another component.

A sixth embodiment can include the method of any of the first to fifth embodiments, further comprising preventing one or more elements from entering the interior of the housing through the inlet via a breathable membrane aligned with the inlet.

A seventh embodiment can include the method of any of the first to sixth embodiments, wherein the breathable membrane comprises a waterproof breathable membrane configured to allow airflow through the membrane while preventing particulate matter and liquids from penetrating the membrane into the interior of the housing.

An eighth embodiment can include the method of any of the first to seventh embodiments, wherein the vents comprise a plurality of openings, wherein each of the plurality of openings aligns with a portion of the breathable membrane.

A ninth embodiment can include the method of any of the first to eighth embodiments, further comprising preventing liquids from entering the interior of the housing through the at least one vent via the breathable membrane.

In a tenth embodiment, a gas detector may comprise a housing configured to enclose one or more components of the gas detector, the housing comprising an inlet and at least one vent; at least one sensor located within the housing, and in fluid communication with the inlet of the housing, configured to detect one or more characteristics of the ambient environment; a fluid channel configured to isolate the sensor from the at least one vent; and at least one breathable membrane located adjacent to the at least one vent, configured to prevent one or more elements from entering the interior of the housing through the at least one vent, wherein when the internal pressure of the housing increases, airflow is directed through the at least one vent to balance the internal pressure with the external pressure of the housing, and wherein when the internal pressure of the housing decreases, airflow is directed through the at least one vent to balance the internal pressure with the external pressure of the housing.

An eleventh embodiment can include the gas detector of the tenth embodiment, further comprising a controller configured to communicate with the at least one sensor.

A twelfth embodiment can include the gas detector of the tenth or eleventh embodiments, wherein the breathable membrane comprises a waterproof breathable membrane configured to allow airflow through the membrane while preventing particulate matter and liquids from penetrating the membrane into the interior of the housing.

A thirteenth embodiment can include the gas detector of any of the tenth to twelfth embodiments, further comprising a layer of adhesive configured to attach the breathable membrane to the housing, wherein at least a portion of the breathable membrane is free of contact with the adhesive.

A fourteenth embodiment can include the gas detector of the thirteenth embodiment, wherein the layer of adhesive is located around the edges of the breathable membrane.

A fifteenth embodiment can include the gas detector of any of the tenth to fourteenth embodiments, wherein the gas detector is configured to detect methane.

In a sixteenth embodiment, a method for venting internal pressure changes of a gas detector may comprise exposing the gas detector to a sensed environment, wherein the gas detector comprises a housing comprising an inlet and at least one vent; at least one sensor in fluid communication with the inlet of the housing; a fluid channel configured to isolate the sensor from the at least one vent; and at least one breathable membrane located adjacent to the at least one vent; preventing one or more elements from entering the interior of the housing through the at least one vent via the breathable membrane; determining one or more characteristics of the sensed environment via the at least one sensor; and when the internal pressure of the housing changes, balancing the change in pressure with the pressure of the external environment via the at least one vent.

A seventeenth embodiment can include the method of the sixteenth embodiment, further comprising preventing one or more elements from entering the interior of the housing through the inlet via a breathable membrane aligned with the inlet.

An eighteenth embodiment can include the method of the sixteenth or seventeenth embodiments, further comprising preventing liquids from entering the interior of the housing through the at least one vent via the breathable membrane.

A nineteenth embodiment can include the method of any of the sixteenth to eighteenth embodiments, further comprising when the internal pressure of the housing increases, directly airflow out of the housing though the at least one vent, and when the internal pressure of the housing decreases, directing airflow into the housing via the at least one vent.

A twentieth embodiment can include the method of any of the sixteenth to nineteenth embodiments, wherein the gas detector further comprises a controller, the method further comprising receiving, by the controller, sensed data from the at least one sensor, and processing, by the controller, the sensed data to determine one or more characteristics of the sensed environment.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present invention(s). Furthermore, any advantages and features described above may relate to specific embodiments, but shall not limit the application of such issued claims to processes and structures accomplishing any or all of the above advantages or having any or all of the above features.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure. Specifically and by way of example, although the headings might refer to a “Field,” the claims should not be limited by the language chosen under this heading to describe the so-called field. Further, a description of a technology in the “Background” is not to be construed as an admission that certain technology is prior art to any invention(s) in this disclosure. Neither is the “Summary” to be considered as a limiting characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of the claims shall be considered on their own merits in light of this disclosure, but should not be constrained by the headings set forth herein.

Use of broader terms such as “comprises,” “includes,” and “having” should be understood to provide support for narrower terms such as “consisting of,” “consisting essentially of,” and “comprised substantially of.” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method for venting internal pressure changes of a gas detector, the method comprising:
providing an inlet to a sensor located within a housing of the gas detector;
isolating the sensor from the rest of the interior of the housing;
providing at least one vent between the interior of the housing and the external environment, wherein the sensor is isolated from airflow into and out of the vent; and
preventing one or more elements from entering the interior of the housing through the at least one vent via a breathable membrane aligned with the at least one vent.

2. The method of claim 1, further comprising:
employing the gas detector in an increased temperature environment;
increasing the internal pressure within the housing due to the increased temperature; and
balancing the increased pressure within the housing with the pressure of the external environment via the at least one vent.

3. The method of claim 1, further comprising employing the gas detector in an increased pressure environment; and balancing the increased pressure within the housing with the pressure of the external environment via the at least one vent.

4. The method of claim 1, further comprising locating the at least one vent and the inlet on a first surface of the housing.

5. The method of claim 4, wherein a second surface of the housing is attached to another component.

6. The method of claim 1, further comprising preventing one or more elements from entering the interior of the housing through the inlet via a breathable membrane aligned with the inlet.

7. The method of claim 1, wherein the breathable membrane comprises a waterproof breathable membrane configured to allow airflow through the membrane while preventing particulate matter and liquids from penetrating the membrane into the interior of the housing.

8. The method of claim 1, wherein the vents comprise a plurality of openings, wherein each of the plurality of openings aligns with a portion of the breathable membrane.

9. The method of claim 1, further comprising preventing liquids from entering the interior of the housing through the at least one vent via the breathable membrane.

10. A gas detector comprising:
a housing configured to enclose one or more components of the gas detector, the housing comprising an inlet and at least one vent;
at least one sensor located within the housing, and in fluid communication with the inlet of the housing, configured to detect one or more characteristics of the ambient environment;
a fluid channel configured to isolate the sensor from the at least one vent; and
at least one breathable membrane located adjacent to the at least one vent, configured to prevent one or more elements from entering the interior of the housing through the at least one vent,
wherein:
when the internal pressure of the housing increases, airflow is directed through the at least one vent to balance the internal pressure with the external pressure of the housing; and
when the internal pressure of the housing decreases, airflow is directed through the at least one vent to balance the internal pressure with the external pressure of the housing.

11. The gas detector of claim 10, further comprising a controller configured to communicate with the at least one sensor.

12. The gas detector of claim 10, wherein the breathable membrane comprises a waterproof breathable membrane configured to allow airflow through the membrane while preventing particulate matter and liquids from penetrating the membrane into the interior of the housing.

13. The gas detector of claim 10, further comprising a layer of adhesive configured to attach the breathable membrane to the housing, wherein at least a portion of the breathable membrane is free of contact with the adhesive.

14. The gas detector of claim 13, wherein the layer of adhesive is located around the edges of the breathable membrane.

15. The gas detector of claim 10, wherein the at least one vent comprises a plurality of vents.

16. A method for venting internal pressure changes of a gas detector, the method comprising:
exposing the gas detector to a sensed environment, wherein the gas detector comprises:
a housing comprising an inlet and at least one vent;
at least one sensor in fluid communication with the inlet of the housing;
a fluid channel configured to isolate the sensor from the at least one vent; and
at least one breathable membrane located adjacent to the at least one vent;
preventing one or more elements from entering the interior of the housing through the at least one vent via the breathable membrane;
determining one or more characteristics of the sensed environment via the at least one sensor; and
when the internal pressure of the housing changes, balancing the change in pressure with the pressure of the external environment via the at least one vent.

17. The method of claim 16, further comprising preventing one or more elements from entering the interior of the housing through the inlet via a breathable membrane aligned with the inlet.

18. The method of claim 16, further comprising preventing liquids from entering the interior of the housing through the at least one vent via the breathable membrane.

19. The method of claim 16, further comprising when the internal pressure of the housing increases, directly airflow out of the housing though the at least one vent, and when the internal pressure of the housing decreases, directing airflow into the housing via the at least one vent.

20. The method of claim 16, wherein the gas detector further comprises a controller, the method further comprising receiving, by the controller, sensed data from the at least one sensor, and processing, by the controller, the sensed data to determine one or more characteristics of the sensed environment.
